(19) United States
(12) Patent Application Publication
(10) Pub. No.: US 2022/0304748 A1
(43) Pub. Date: Sep. 29, 2022

(54) CATHETER DEVICE, CATHETER BODY, AND CATHETER

(71) Applicant: Japan Lifeline Co.,Ltd., Tokyo (JP)

(72) Inventor: Kohei SAKAKI, Tokyo (JP)

(21) Appl. No.: 17/840,888

(22) Filed: Jun. 15, 2022

Related U.S. Application Data
(63) Continuation of application No. PCT/JP2020/011605, filed on Mar. 17, 2020.

(51) Int. Cl. A61B 18/14; A61B 5/01

(52) U.S. Cl. CPC A61B 18/1492; A61B 5/01; A61B 2018/00577

(57) ABSTRACT

A catheter device is to be applied to a catheter and includes a tube member and a second handle. The tube member is configured to be inserted through a lumen provided in a catheter shaft of the catheter. The second handle is provided on a base end of the tube member, and includes a deformation operating member. The second handle is provided separately from a first handle provided on a base end of the catheter shaft. Upon bending deformation of a region in the vicinity of a tip end of the tube member in response to an operation of the deformation operating member, the tube member is configured to be pressed against a wall surface of the lumen of the catheter shaft to displace a region in the vicinity of a tip end of the catheter shaft. The second handle is configured to be integrated with the first handle.

### CROSS REFERENCE TO RELATED APPLICATIONS

This is a continuation of International Application No. PCT/JP2020/11605, filed Mar. 17, 2020, the entire contents of which are incorporated herein by reference.

### BACKGROUND

The technology relates to a catheter to be used for measuring an internal temperature of a hollow organ inside the body such as the esophagus, and to a catheter body and a catheter device each of which is to be applied to the catheter.

An operation that performs cauterization or “ablation” with use of an ablation catheter has been performed as one of medical treatments for arrhythmia, etc. Such ablation that uses the ablation catheter may be performed on a site that involves the arrhythmia inside the heart, for example. In general, methods of the ablation may be roughly classified into a method that performs heating and a method that performs cooling. For example, the methods of the ablation may be roughly classified into a high-temperature ablation that uses a high frequency current and a low-temperature ablation that uses liquid nitrous oxide, liquid nitrogen, etc. Upon performing the ablation of a site such as the posterior wall of the left atrium of the heart by means of the ablation catheter, i.e., upon surgical ablation of the left atrium, the esophagus positioned in the vicinity of the posterior wall of the left atrium can typically be heated or cooled as well, leading to a possible damage of the esophagus.

To address this, a method has been proposed that measures or monitors data on a temperature in the esophagus, such as a temperature of the medial wall of the esophagus. The method involves insertion of a temperature measuring catheter or a so-called “esophageal catheter” into the esophagus through the nose of a patient by means of a transnasal approach. For example, reference is made to Japanese Unexamined Patent Application Publication (Translation of PCT Application) No. 2010-505592. Monitoring the temperature in the esophagus makes it possible to avoid a possible damage of the esophagus upon, for example, the surgical ablation of the left atrium described above.

### SUMMARY

A catheter device according to one embodiment of the technology is to be applied to a catheter. The catheter includes a catheter shaft, and a plurality of temperature sensors provided in a region in the vicinity of a tip end of the catheter shaft and configured to measure an internal temperature of a hollow organ inside the body. The catheter device includes a tube member and a second handle. The tube member extends in an axial direction, and is configured to be inserted through a lumen provided in the catheter shaft. The second handle is provided on a base end of the tube member, and includes a deformation operating member configured to receive an operation that causes a region in the vicinity of a tip end of the tube member to be subjected to bending deformation. The second handle is provided separately from a first handle provided on a base end of the catheter shaft. Upon the bending deformation of the region in the vicinity of the tip end of the tube member in response to the operation of the deformation operating member, the tube member to be subjected to the bending deformation is configured to be pressed against a wall surface of the lumen of the catheter shaft to displace the region in the vicinity of the tip end of the catheter shaft. The second handle is configured to be integrated with the first handle.

A catheter body according to one embodiment of the technology is a catheter body of a catheter configured to measure an internal temperature of a hollow organ inside the body. The catheter body includes a catheter shaft, a plurality of temperature sensors, and a first handle. The catheter shaft has a lumen. The plurality of temperature sensors is provided in a region in the vicinity of a tip end of the catheter shaft, and is configured to measure the internal temperature of the hollow organ inside the body. The first handle is provided on a base end of the catheter shaft. Upon bending deformation of a region in the vicinity of a tip end of a tube member of a catheter device in response to an operation of a deformation operating member of the catheter device, the tube member to be subjected to the bending deformation is configured to be pressed against a wall surface of the lumen of the catheter shaft to displace the region in the vicinity of the tip end of the catheter shaft. The catheter device includes the tube member and a second handle. The tube member extends in an axial direction, and is configured to be inserted through the lumen of the catheter shaft. The second handle is provided on a base end of the tube member, and includes the deformation operating member configured to receive the operation that causes the region in the vicinity of the tip end of the tube member to be subjected to the bending deformation. The second handle is provided separately from the first handle. The first handle is configured to be integrated with the second handle.

A catheter according to one embodiment of the technology is configured to measure an internal temperature of a hollow organ inside the body. The catheter includes a catheter body and a catheter device. The catheter body includes a catheter shaft, a plurality of temperature sensors, and a first handle. The catheter shaft has a lumen. The plurality of temperature sensors is provided in a region in the vicinity of a tip end of the catheter shaft, and configured to measure the internal temperature of the hollow organ inside the body. The first handle is provided on a base end of the catheter shaft. The catheter device includes a tube member and a second handle. The tube member extends in an axial direction, and is inserted through the lumen of the catheter shaft. The second handle is provided on a base end of the tube member, and includes a deformation operating member configured to receive an operation that causes a region in the vicinity of a tip end of the tube member to be subjected to bending deformation. Upon the bending deformation of the region in the vicinity of the tip end of the tube member in response to the operation of the deformation operating member, the tube member to be subjected to the bending deformation is configured to be pressed against a wall surface of the lumen of the catheter shaft to displace the region in the vicinity of the tip end of the catheter shaft. The first handle and the second handle are configured to be integrated with each other.

It is to be understood that both the foregoing general description and the following detailed description are exemplary, and are intended to provide further explanation of the technology as claimed.

### BRIEF DESCRIPTION OF THE DRAWINGS

The accompanying drawings are included to provide a further understanding of the disclosure, and are incorporated in and constitute a part of this specification. The drawings illustrate embodiments and, together with the specification, serve to explain the principles of the technology.

FIG. 1 is a diagram schematically illustrating an example of an outline configuration of a catheter according to one example embodiment of the technology.

FIG. 2 is a diagram schematically illustrating integration of two handles illustrated in FIG. 1.

FIG. 3 is another diagram schematically illustrating the integration of two handles illustrated in FIG. 1.

FIG. 4 is a cross-sectional diagram taken along line IV-IV illustrated in FIG. 1 and seen in a direction of arrows in FIG. 1.

FIG. 5 is a diagram schematically illustrating an example of an internal structure of the handle of a catheter body illustrated in FIG. 1.

FIG. 6 is another diagram schematically illustrating an example of the internal structure of the handle of the catheter body illustrated in FIG. 1.

FIG. 7A is a diagram schematically illustrating an example of a detailed configuration of a region in the vicinity of a tip end of a tube member illustrated in FIG. 1.

FIG. 7B is a diagram schematically illustrating an example of operation upon bending deformation of the region in the vicinity of the tip end of the tube member illustrated in FIG. 1.

FIG. 8 is a diagram schematically illustrating an example of an internal structure of the handle of a catheter device illustrated in FIG. 1.

FIGS. 9A and 9B are each a diagram schematically illustrating an example of operation of the handle of the catheter device illustrated in FIG. 8.

FIGS. 10A and 10B are each a diagram schematically illustrating an example of how the catheter illustrated in FIG. 1 is used.

FIG. 11A is a diagram schematically illustrating an example of a configuration of an opening of the tube member according to one example embodiment.

FIG. 11B is a diagram schematically illustrating an example of a configuration of the opening of a tube member according to modification example 1-1.

FIG. 11C is a diagram schematically illustrating an example of a configuration of the opening of a tube member according to modification example 1-2.

FIG. 11D is a diagram schematically illustrating an example of a configuration of the opening of a tube member according to modification example 1-3.

FIGS. 12A and 12B are each a diagram schematically illustrating an example of a configuration of slits of a tube member according to modification example 2.

FIG. 13A is a diagram schematically illustrating an example of a configuration of the slits according to the modification example 2, where the slits are deployed on a plane.

FIG. 13B is a diagram schematically illustrating an example of a configuration of the slits according to modification example 3-1, where the slits are deployed on a plane.

FIG. 13C is a diagram schematically illustrating an example of a configuration of the slits according to modification example 3-2, where the slits are deployed on a plane.

FIG. 14A is a diagram schematically illustrating an example of a configuration of a metal member, of the tube member, that uses a metal line according to one example embodiment.

FIG. 14B is a diagram schematically illustrating an example of a configuration of a metal member, of the tube member, that uses the metal line according to modification example 4-1.

FIG. 14C is a diagram schematically illustrating an example of a configuration of a metal member, of the tube member, that uses the metal line according to modification example 4-2.

### DETAILED DESCRIPTION

In general, it is demanded that a catheter be able to reduce a burden to be imposed on the body of a patient while more reliably preventing a possibility of a damage of a hollow organ inside the body such as the esophagus upon measuring an internal temperature of the hollow organ, and to reduce a cost upon use of the catheter.

It is desirable to provide a catheter that makes it possible to reduce a burden to be imposed on the body of a patient while more reliably preventing a possibility of a damage of a hollow organ inside the body upon measuring an internal temperature of the hollow organ, and to reduce a cost upon use, and a catheter body and a catheter device each of which is to be applied to the catheter.

Some example embodiments of the technology are described in detail below, in the following order, with reference to the drawings.

### 1. Example Embodiment (an Example of a Configuration of a Catheter that Includes a Catheter Body and a Catheter Device)

### 2. Modification Examples

Modification Examples 1 (other examples of a configuration of an opening in a region the vicinity of a tip end of a tube member)

Modification Examples 2 and 3 (examples of a configuration in which slits are provided in the region in the vicinity of the tip end of a tube member)

Modification Examples 4 (other examples of a metal line of a tube member)

Note that the following description is directed to illustrative examples of the technology and not to be construed as limiting to the technology. Factors including, without limitation, numerical values, shapes, materials, components, positions of the components, and how the components are coupled to each other are illustrative only and not to be construed as limiting to the technology. Further, elements in the following example embodiments which are not recited in a most-generic independent claim of the technology are optional and may be provided on an as-needed basis. The drawings are schematic and are not intended to be drawn to scale. Note that the like elements are denoted with the same reference numerals, and any redundant description thereof will not be described in detail.

### 1. Example Embodiment

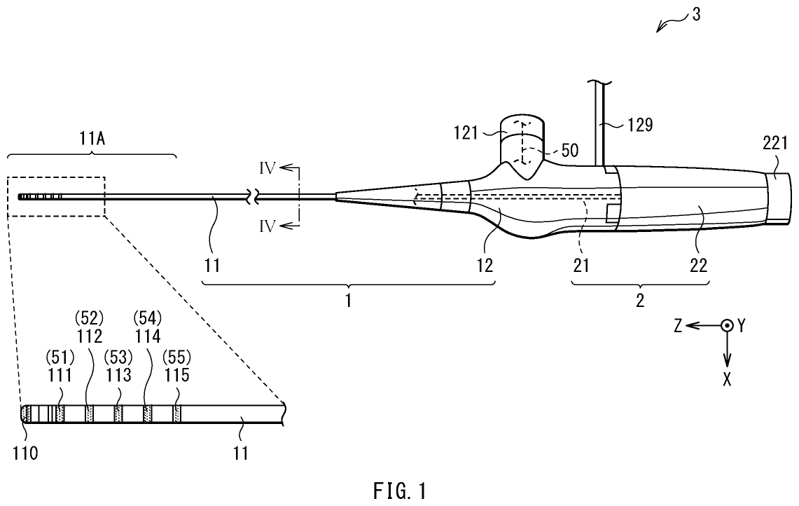

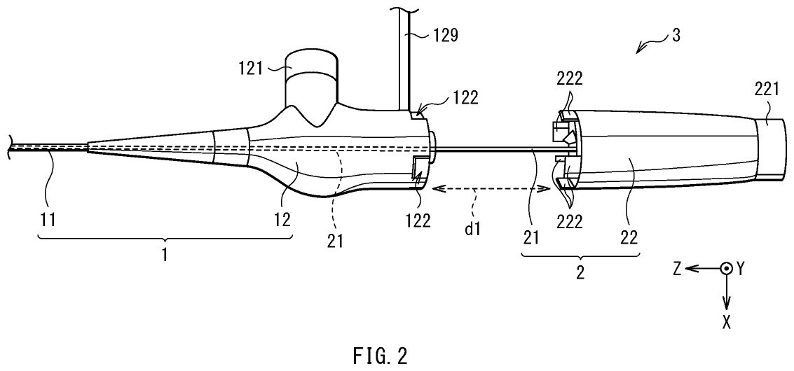

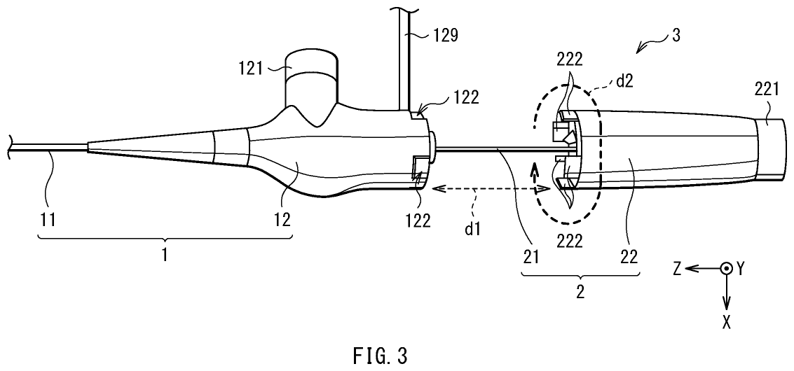

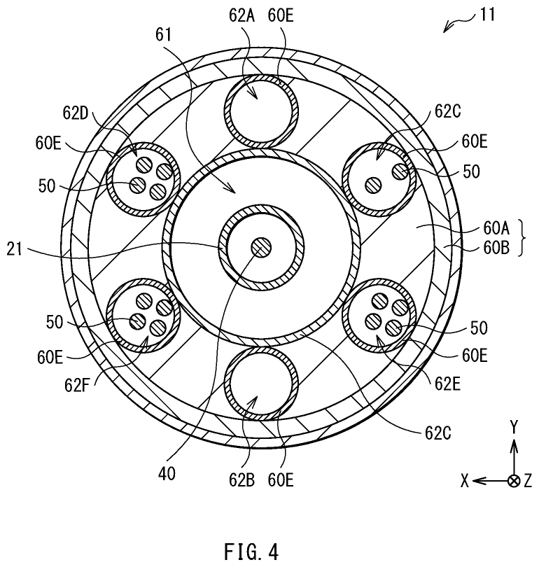

FIG. 1 is a front diagram schematically illustrating an example of an outline configuration, on a Z-X plane, of a catheter 3 according to an example embodiment of the technology. FIGS. 2 and 3 are schematic diagrams each illustrating integration of later-described two handles 12 and 22 illustrated in FIG. 1. FIG. 4 is a cross-sectional diagram of a configuration, on a X-Y plane, taken along line IV-IV illustrated in FIG. 1 and seen in a direction of arrows in FIG. 1.

The catheter 3 may be a catheter or a so-called “esophageal catheter” to be used for a measurement of data on an internal temperature of a hollow organ inside the body of a patient upon performing a medical treatment of, for example, arrhythmia of the patient, e.g., upon performing surgical ablation of the left atrium. For example, the hollow organ may be the digestive system such as the esophagus. The internal temperature may be a temperature of the medial wall of the hollow organ. For example, the catheter 3 may be inserted into the esophagus, or any other part, of the patient through the nose or the “nasal cavity” by means of a transnasal approach, as described later in greater detail. Alternatively, the catheter 3 may be inserted into the esophagus, or any other part, of the patient through the mouth by means of a peroral approach.

Referring to FIG. 1, the catheter 3 includes a catheter body 1. The catheter 3 includes a catheter device 2 to be applied to the catheter 3. The catheter body 1 may be a single-use or disposable device to be disposed each time a patient is treated, whereas the catheter device 2 may be a reusable device that allows for reuse after the treatment of the patient.

### Catheter Body 1

As illustrated in FIGS. 1 to 3, the catheter body 1 includes: a catheter shaft 11 or a “catheter tube” serving as an elongated part; and a handle 12 provided on a base end of the catheter shaft 11.

The handle 12 may correspond to a specific but non-limiting example of a “first handle” according to one embodiment of the disclosure.

### Catheter Shaft 11

The catheter shaft 11 may have a tubular structure having flexibility, and may have a shape that extends in an axial direction thereof, i.e., in a Z-axis direction. In other words, the catheter shaft 11 may be a hollow tube-shaped member. For example, a length in the axial direction of the catheter shaft 11 may be about several times to about several ten times as long as a length in an axial direction, i.e., in the Z-axis direction, of the handle 12.

As illustrated in FIG. 1, the catheter shaft 11 may have a tip end part, or a “tip-flexible part 11A”, that has a relatively superior flexibility. The catheter shaft 11 may also have a so-called multiple-lumen structure in which a plurality of lumens is so formed therein as to extend in the axial direction thereof, i.e., in the Z-axis direction. The term “lumen” as used herein may encompass an inner hole, a pore, or a through hole. The lumen provided in the catheter shaft 11 may include various fine wires, such as electrical leads 50 to be described later, that are inserted therethrough while they are electrically insulated from one another. The lumen may also include a tube member 21 of the later-described catheter device 2 inserted therethrough.

For example, as illustrated in FIG. 4, the catheter shaft 11 may have: one main lumen 61 disposed in the middle of the catheter shaft 11; and a plurality of sub-lumens 62A to 62F isotropically disposed on an outer circumferential side of the main lumen 61. FIG. 4 illustrates an example in which the catheter shaft 11 has six sub-lumens 62A to 62F, although the number of sub-lumens is not limited thereto.

The main lumen 61 may correspond to a specific but non-limiting example of a “lumen” according to one embodiment of the disclosure.

The main lumen 61 may include the tube member 21 of the catheter device 2. The tube member 21 extends in the axial direction, i.e., in the Z-axis direction, and is inserted through the main lumen 61. As illustrated in FIG. 4, the main lumen 61 may include an operating wire 40 of the later-described catheter device 2. The operating wire 40 may be inserted through the tube member 21. For example, the main lumen 61 may have an inner diameter in a range from about 0.6 mm to about 4.5 mm.

In an example illustrated in FIG. 4, the sub-lumens 62A and 62B each may include no fine wires inserted therethrough. The sub-lumens 62C, 62D, 62E, and 62F each may include the electrical leads 50, or “leads”, inserted therethrough. The fine wires, or the “electrical leads 50”, may extend in the axial direction, i.e., in the Z-axis direction, of the catheter shaft 11.

The electrical leads 50 may have respective tip ends electrically coupled individually to respective electrodes 111 to 115 described later. As illustrated in FIG. 1, the electrical leads 50 may have respective base ends that are connectable to the outside of the catheter 3 from the inside of the catheter shaft 11, i.e., from the inside of the sub-lumens 62C, 62D, 62E, and 62F via the inside of the handle 12 and the inside of a later-described connector 121.

As illustrated in FIG. 4, the catheter shaft 11 may include: a tube 60A positioned on an inner circumferential side of the catheter shaft 11; and a tube 60B positioned on an outer circumferential side of the catheter shaft 11. The main lumen 61 may be formed in a tube 60C positioned on an inner circumferential side of the tube 60A. The sub-lumens 62A to 62F may be formed in respective tubes 60E disposed inside the tube 60A.

The catheter shaft 11 may have an outer diameter in a range from about 1.0 mm to about 5.0 mm, for example. The catheter shaft 11 may have a length in the axial direction in a range from about 300 mm to about 1500 mm. The catheter shaft 11, or the tubes 60A, 60B, 60C, and 60E, may include a thermoplastic resin as a constituent material, such as polyamide, polyether poly amide, polyurethane, polyether block amide, i.e., PEBAX (Registered Trademark), or nylon. The tubes 60C and 60E may include a fluororesin as the thermoplastic resin, such as perfluoroalkoxy alkane (PFA) or polytetrafluoroethylene (PTFE). The tube 60B may have: an outer circumferential layer that includes a resin such as polyamide; and an inner circumferential layer that includes a stainless steel (SUS) braid, for example.

As illustrated in FIG. 1, the plurality of ring-shaped electrodes 111 to 115 and one tip 110 may be disposed at a predetermined interval in the vicinity of a tip end, i.e., the tip-flexible part 11A, of the catheter shaft 11. The electrodes 111 to 115 each may include a metal ring. For example, the electrodes 111 to 115 each may be fixedly disposed at a mid-part of the tip-flexible part 11A, i.e., around a middle region of the tip-flexible part 11A, whereas the tip 110 may be fixedly disposed at a most distal end of the tip-flexible part 11A.

The five electrodes 111 to 115 may be disposed side by side in this order at a predetermined interval from a tip end, i.e., from the tip 110, of the catheter shaft 11 to a base end of the catheter shaft 11. The predetermined interval, or a distance from an electrode to an electrode among the electrodes 111 to 115, may be 10 mm or less, or may be in a range from about 2 mm to about 5 mm, for example. In some embodiments, the predetermined interval may be 5 mm. The electrodes 111 to 115 each may have a width of 7 mm or less, or in a range from about one mm to about 5 mm, for example. In some embodiments, the electrodes 111 to 115 each may have a width of 5 mm.

The electrodes 111 to 115 each may include a metal material having a good electrical conductivity, such as aluminum (Al), copper (Cu), stainless steel (SUS), gold (Au), or platinum (Pt). The tip 110 may include a metal material similar to that of each of the electrodes 111 to 115, for example. Alternatively, the tip 110 may include a resin material such as a silicone rubber resin or polyurethane. An outer diameter of each of the electrodes 111 to 115 and the tip 110 is not particularly limited. In some embodiments, the electrodes 111 to 115 and the tip 110 each may have the outer diameter that is about the same as the outer diameter of the catheter shaft 11.

As denoted by parentheses in FIG. 1, the tip-flexible part 11A of the catheter shaft 11 may be provided therein with five temperature sensors 51 to 55 that are respectively disposed in the vicinity of the electrodes 111 to 115 and disposed corresponding to the electrodes 111 to 115. For example, the temperature sensors 51 and 55 may be provided at positions opposed to the respective electrodes 111 to 115. In this example embodiment, a plurality of sets of electrodes 111 to 115 and temperature sensors 51 to 55, i.e., five sets configured by the five electrodes 111 to 115 and the five temperature sensors 51 to 55, are provided in a one-to-one correspondence relationship. It should be noted that, in this example embodiment, no temperature sensor that forms a pair with, or is electrically coupled to, the tip 110 may be provided in the vicinity of the tip 110.

The temperature sensors 51 to 55 each may serve as a sensor that measures an internal temperature of a site such as the esophagus upon, for example, the surgical ablation of the left atrium. The temperature sensors 51 to 55 may be electrically coupled to the electrodes 111 to 115 in an individual fashion, respectively. For example, as illustrated in FIG. 1, the temperature sensor 51 may be embedded in the vicinity of the electrode 111, and may be electrically coupled to the electrode 111. Similarly, the temperature sensor 52 may be embedded in the vicinity of the electrode 112, and may be electrically coupled to the electrode 112. The temperature sensor 53 may be embedded in the vicinity of the electrode 113, and may be electrically coupled to the electrode 113. The temperature sensor 54 may be embedded in the vicinity of the electrode 114, and may be electrically coupled to the electrode 114. The temperature sensor 55 may be embedded in the vicinity of the electrode 115, and may be electrically coupled to the electrode 115. For example, such electrical coupling may be achieved by an individual spot welding of the temperature sensors 51 to 55 onto corresponding inner circumferential surfaces of the respective electrodes 111 to 115.

The temperature sensors 51 to 55 each may have a configuration in which a thermocouple is used, for example. In other words, the temperature sensors 51 to 55 each may utilize a temperature measuring junction by means of the thermocouple. The leads, or the electrical leads 50 described above, may be electrically coupled to the respective temperature sensors 51 to 55 in an individual fashion, and may include metal wires. The metal wires may be different in kind from each other and structure the thermocouple. As illustrated in FIGS. 1 and 4, the electrical leads 50 each may be inserted through the lumen, i.e., the sub-lumens 62C to 62F, provided in the catheter shaft 11 and led to the inside of the handle 12 as described previously.

### Handle 12

As illustrated in FIGS. 1 to 3, the handle 12 is provided on the base end of the catheter shaft 11, and may serve as a part where an operator such as a doctor grabs or holds upon using the catheter 3, i.e., the catheter body 1. As described later in greater detail, the handle 12 is provided separately from a handle 22 of the later-described catheter device 2.

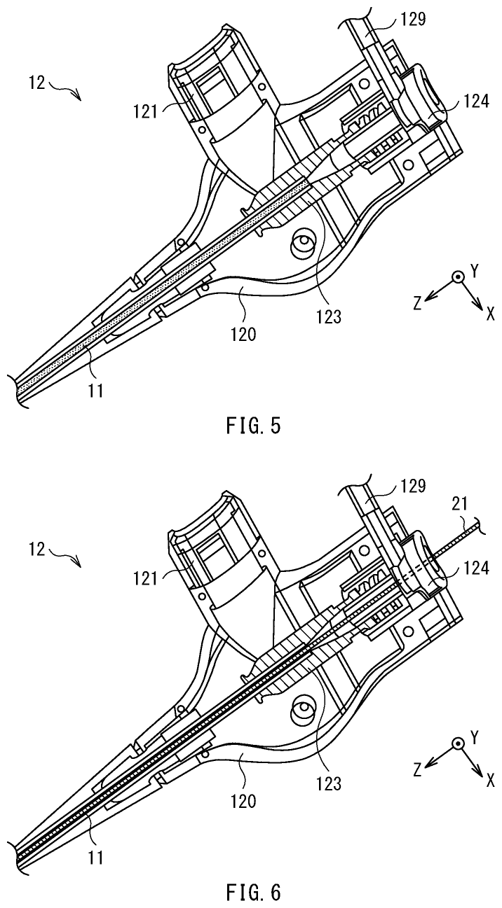

FIGS. 5 and 6 each schematically illustrate an example of an internal structure of the handle 12 of the catheter body 1. FIG. 6 illustrates the internal structure of the handle 12 in which the tube member 21 described above of the catheter device 2 is inserted through the catheter shaft 11 as compared with the internal structure illustrated in FIG. 5.

As illustrated in FIGS. 1 to 3, 5, and 6, the handle 12 may include a handle body 120, a connector 121, a plurality of recesses 122, a guide 123, an insertion hole 124, and a fluid injection tube 129.

The handle body 120 may be equivalent to a part or a “grip” where the operator actually holds, and may also serve as an exterior member of the handle 12. The handle body 120 may include a synthetic resin such as polycarbonate, acrylonitrile butadiene styrene copolymer (ABS), acrylic, polyolefin, polyoxymethylene, or polyacetal.

The connector 121 may allow the electrical leads 50 described above, i.e., the leads electrically coupled to the temperature sensors 51 to 55 in an individual fashion, to be coupled to the outside of the catheter 3. As illustrated in FIGS. 1 to 3, 5, and 6, the connector 121 may be provided in an X-axis direction at a side face, of the handle 12, that is off the axial direction, i.e., the Z-axis direction. For example, the connector 121 may be so provided as to project in the X-axis direction from the handle body 120 that extends in the Z-axis direction.

As illustrated in FIGS. 2 and 3, the recesses 122 each may be provided on a part, of the handle 12, that faces the later-described handle 22, and may extend in the Z-axis direction. The recesses 122 may be disposed circularly on an X-Y plane of the handle body 120. The recesses 122 may be configured to be individually fitted with respective projections 222 provided on the later-described handle 22, as denoted by a broken-line arrow d1 illustrated in FIGS. 2 and 3. The handle 12 is thus configured to be integrated with the later-described handle 22. In other words, the handles 12 and 22 are configured to be integrated with each other, and configured to be divided into two handles 12 and 22 separate from each other with respect to a handle as a whole.

As illustrated in FIGS. 5 and 6, the guide 123 may fix a base-end part of the catheter shaft 11 inside the handle body 120. As illustrated in FIG. 6, the guide 123 may guide an insertion path of the catheter shaft 11 upon insertion of the later-described tube member 21 into the catheter shaft 11.

As illustrated in FIG. 6, the insertion hole 124 may receive the insertion of the later-described tube member 21 into the handle body 120. The insertion hole 124 may be configured to fix a position of the tube member 21 being inserted into the handle body 120, i.e., a position of the insertion of the tube member 21 into the catheter shaft 11. For example, the insertion hole 124 may be configured by a rubber valve.

As illustrated in FIGS. 5 and 6, the fluid injection tube 129 may allow a predetermined fluid, such as a contrast medium, to be injected from the inside of the handle body 120 into the catheter shaft 11. The fluid thus injected from the fluid injection tube 129 may travel through the inside of the main lumen 61 of the catheter shaft 11, following which the fluid may be discharged to the outside via a through hole provided on the tip 110 described above.

### Catheter Device 2

As illustrated in FIGS. 1 to 3, the catheter device 2 includes: the tube member 21, or a “stylet”, as an elongated part; and the handle 22 provided on a base end of the tube member 21.

The handle 22 may correspond to a specific but non-limiting example of a “second handle” according to one embodiment of the disclosure. The handle 22 may correspond to a specific but non-limiting example of a “handle” according to one embodiment of the disclosure.

### Tube Member 21

As illustrated in FIGS. 1 to 3 and 6, the tube member 21 is inserted into the lumen, i.e., the main lumen 61, provided in the catheter shaft 11 of the catheter body 1 described above. The tube member 21 extends in the axial direction, i.e., the Z-axis direction. As illustrated in FIG. 6, the tube member 21 may be inserted into the catheter shaft 11, i.e., into the main lumen 61, with the inside of the handle 12, i.e., the inside of the handle body 120, being linearly inserted in the Z-axis direction.

The tube member 21 may have an outer diameter in a range from about 0.5 mm to about 4.0 mm, for example. The tube member 21 may have a length in the axial direction in a range from about 400 mm to about 1700 mm, for example.

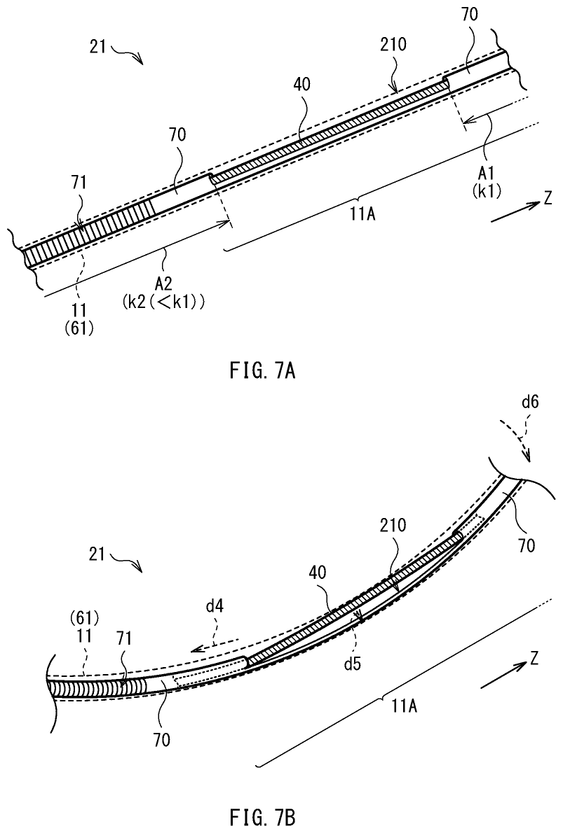

FIGS. 7A and 7B each schematically illustrate an example of details of the tube member 21. FIG. 7A schematically illustrates an example of a detailed configuration of a region in the vicinity of a tip end of the tube member 21. FIG. 7B schematically illustrates an example of operation upon bending deformation of the region in the vicinity of the tip end of the tube member 21. It should be noted that the catheter shaft 11, i.e., a region in the vicinity of the tip-flexible part 11A, is also illustrated by a broken line in FIGS. 7A and 7B.

Referring to FIGS. 7A and 7B, the operating wire 40 extending in the Z-axis direction may be inserted through the tube member 21. The operating wire 40 may have a tip end fixed to a tip-end part of the tube member 21, and a base end fixed to the inside of the later-described handle 22. Further, as illustrated in FIGS. 7A and 7B, the tube member 21 may have an opening 210 provided in the region in the vicinity of the tip end of the tube member 21, i.e., provided in the vicinity of the tip-flexible part 11A of the catheter shaft 11. The opening 210 may be rectangular in shape, and may have a longitudinal direction that extends in the axial direction, i.e., the Z-axis direction. In other words, the tube member 21 may have, in the region in the vicinity of the tip end of the tube member 21, a halved structure having the opening 210.

For example, “the region in the vicinity of the tip end” of the tube member 21 may refer to a part having a length extending by (⅓)×L from a region near the tip end of the tube member 21 to the base end of the tube member 21, where “L” is an overall length from the tip end of the tube member 21 to the base end of the tube member 21. In other words, “the region in the vicinity of the tip end” of the tube member 21 may refer to a part having the length extending by (⅓)×L from a position, distant from the tip end of the tube member 21 toward the base end of the tube member 21 by a predetermined distance, to the base end of the tube member 21. However, “the region in the vicinity of the tip end” of the tube member 21 is not limited to the definition described above, and may be defined by any other definition.

As illustrated in FIGS. 7A and 7B, the tube member 21 may be configured by a metal member 70. The metal member 70 may be configured by a metal pipe, for example. At least a distal end in the axial direction, i.e., the Z-axis direction, of the metal member 70 may have a single metal coil 71 that serves as one or more spirally wound metal lines. In an example illustrated in FIGS. 7A and 7B, only the base end of the metal member 70 may have the metal coil 71. In other words, in an example illustrated in FIGS. 7A and 7B, the base end of the metal member 70 may be provided with the metal coil 71, whereas the tip end in the axial direction of the metal member 70 may not be provided with the metal coil 71. It should be noted that a configuration of the metal member 70 is not limited thereto. In some embodiments, both the base end and the tip end of the metal member 70 may be provided with the metal coil 71 that serves as the metal line.

The metal coil 71 may correspond to a specific but non-limiting example of a “metal line” according to one embodiment of the disclosure.

The metal member 70 and the metal coil 71 each may include a metal material such as a stainless-steel alloy or a nickel-titanium alloy.

As illustrated in FIG. 7A, a region on the tip end side and a region on the base end side of the region in the vicinity of the tip end of the tube member 21 may have the following example magnitude relationship in terms of rigidity, with reference to the region in the vicinity of the tip end of the tube member 21 (see the vicinity of the opening 210 illustrated in FIG. 7B). Namely, in an example of FIG. 7A, a rigidity k1 of the tube member 21 in a region A1 that is on the tip end side of a region near the opening 210 may be greater than a rigidity k2 of the tube member 21 in a region A2 that is on the base end side of the region near the opening 210 (k1>k2). The region in the vicinity of the tip end of the tube member 21 serves as a part to be subjected to bending deformation as described later.

The bending deformation, or a bending deformation operation, of the region in the vicinity of the tip end of the tube member 21 illustrated in FIG. 7B will be described later in greater detail.

### Handle 22

As illustrated in FIGS. 1 to 3, the handle 12 is provided on the base end of the tube member 21, and may serve as a part where an operator such as the doctor grabs or holds upon using the catheter 3, i.e., the catheter device 2. As described later in greater detail, the handle 22 is provided separately from the handle 12 of the catheter body 1 described above.

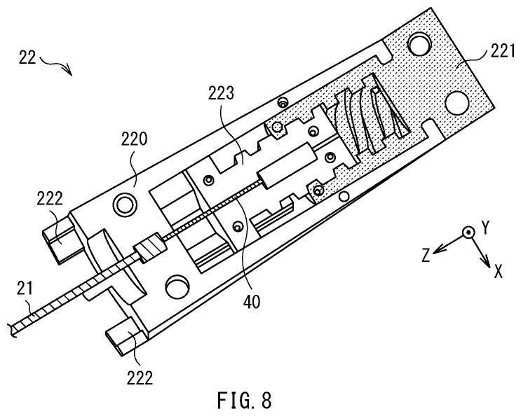

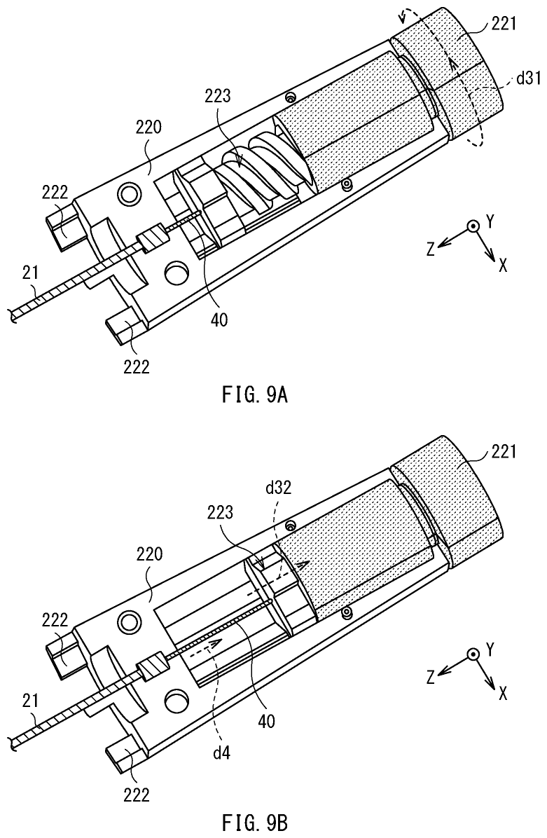

FIG. 8 schematically illustrates an example of an internal structure of the handle 22 of the catheter device 2. FIGS. 9A and 9B each schematically illustrate an example of operation of the handle 22 illustrated in FIG. 8.

As illustrated in FIGS. 1 to 3, 8, 9A, and 9B, the handle 22 may include a handle body 220, a rotary member 221, a plurality of protrusions 222, and a drive member 223.

The handle body 220 may be equivalent to a part or a “grip” where the operator actually holds, and may also serve as an exterior member of the handle 22. As illustrated in FIGS. 8, 9A, and 9B, the base end of the tube member 21 may be fixed on the handle body 220. The handle body 220 may include a synthetic resin similar to that of the handle body 120 described above, for example.

As illustrated in FIGS. 2, 3, 8, 9A, and 9B, the protrusions 222 each may be provided on a part, of the handle 22, that faces the handle 12 described above, and may extend in the Z-axis direction. The protrusions 222 may be disposed circularly on an X-Y plane of the handle body 220. The protrusions 222 may be configured to be individually fitted with the respective recesses 122 provided on the handle 12 described above, as denoted by the broken-line arrow d1 illustrated in FIGS. 2 and 3. The handle 22 is thus configured to be integrated with the above-described handle 12. In other words, as described above, the handles 12 and 22 are configured to be integrated with each other, and configured to be divided into two handles 12 and 22 separate from each other with respect to a handle as a whole.

As denoted by a broken-line arrow d2 illustrated in FIG. 3, optionally, it is possible to perform the following example adjustment on the basis of an angle of integration within a plane, i.e., an X-Y plane, orthogonal to the axial direction, i.e., the Z-axis direction, upon the integration of the handles 12 and 22 with each other. Namely, it is possible to adjust, to any orientation, a later-described orientation of deformation upon the bending deformation of the region in the vicinity of the tip end of the tube member 21 as illustrated in FIG. 7B, on the basis of the angle of integration of the handles 12 and 22 with each other. For example, changing a combination of mutual fitting of the respective recesses 122 of the handle 12 and the respective protrusions 222 of the handle 22 makes it possible to also change the angle of integration described above and thereby to change the orientation of deformation upon the bending deformation described above to a desired orientation as well.

As illustrated in FIGS. 8, 9A, and 9B, the rotary member 221 may be disposed at a base end part of the handle 22, i.e., at a base end of the handle body 220. The rotary member 221 serves as an operating part where an operation, e.g., a “rotating operation”, that causes the region in the vicinity of the tip end of the tube member 21 to be subjected to the bending deformation is to be performed by an operator. In other words, the rotary member 221 may be a part to be used upon the rotating operation.

The rotary member 221 may correspond to a specific but non-limiting example of a “deformation operating member” according to one embodiment of the disclosure.

The drive member 223 may move bidirectionally in the axial direction, i.e., the Z-axis direction, inside the handle body 220 in response to the above-described rotating operation performed on the rotary member 221. As illustrated in FIGS. 8, 9A, and 9B, the base end of the operating wire 40 described above may be fixed on the drive member 223 inside the handle body 220. The drive member 223 may thus drive the operating wire 40 as described below.

In a case where the rotary member 221 of the handle 22 thus configured is subjected to the rotating operation by an operator as denoted by a broken-line arrow d31 illustrated in FIG. 9A, the drive member 223 may move inside the handle body 220 as denoted by a broken-line arrow d32 illustrated in FIG. 9B in response to the rotating operation. For example, the drive member 223 may move toward the rotary member 221, i.e., toward the base end, in the Z-axis direction inside the handle body 220. Accordingly, the operating wire 40 may be pulled toward the base end as denoted by a broken-line arrow d4 illustrated in FIG. 9B, thereby causing the region in the vicinity of the tip end of the tube member 21 to be subjected to the bending deformation as described later in greater detail.

### Operation, Workings, and Effects

### A. Basic Operation

The catheter 3 may allow for measurement of data on the internal temperature of a hollow organ inside the body, such as the esophagus, of a patient when being used for a medical treatment of arrhythmia, etc., of the patient, e.g., when being used for surgical ablation of the left atrium. For example, the catheter body 1 of the catheter 3 may be used to measure the data on the internal temperature of the hollow organ. Examples of the ablation to be performed upon the medical treatment may include a high-temperature ablation, i.e., a heating method, that uses a high frequency current and a low-temperature ablation, i.e., a cooling method, that uses liquid nitrous oxide, liquid nitrogen, etc.

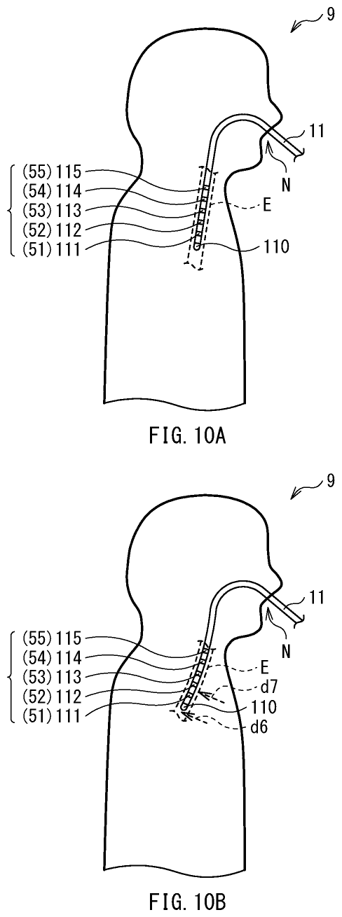

As schematically illustrated in FIG. 10A, the catheter shaft 11 of the catheter body 1 may be inserted from the tip end, i.e., from the tip-flexible part 11A, of the catheter shaft 11 into the esophagus E of a patient 9 through, for example, the nose or the “nasal cavity N” of the patient 9 by means of a transnasal approach, upon performing the measurement of the internal temperature.

The tip-flexible part 11A of the catheter shaft 11 may include the five electrodes 111 to 115 serving as the temperature measuring metal rings. The tip-flexible part 11A may also include the five temperature sensors 51 to 55 electrically coupled to the electrodes 111 to 115 in an individual fashion, respectively. Utilizing the electrodes 111 to 115 and the temperature sensors 51 to 55 allows for measurement or monitoring of the data on the internal temperature of the esophagus E. It should be noted that, as illustrated in FIG. 10A, the electrode 111 and the electrode 115 may respectively be so disposed as to measure the lower side and the upper side of the esophagus when the catheter shaft 11 is inserted, from the tip-flexible part 11A of the catheter shaft 11, into the esophagus E of the patient 9. The lower side and the upper side may respectively be, in other words, the stomach side and the oral cavity side.

Monitoring the internal temperature of the esophagus E of the patient 9 by means of the catheter body 1 helps to avoid a possibility that the esophagus E is damaged upon, for example, the foregoing surgical ablation of the left atrium. For example, when performing ablation of a site such as the posterior wall of the left atrium of the heart by means of an ablation catheter, i.e., upon the surgical ablation of the left atrium, the esophagus located in the vicinity of the posterior wall of the left atrium can typically be heated or cooled as well, leading to a possible damage of the esophagus. Monitoring the internal temperature of the esophagus E as described above makes it possible to take a precaution and thus helps to avoid the possibility of the damage.

For example, it is possible to take measures to cut off a supply of electricity to the ablation catheter, i.e., the catheter body 1, in a case where the internal temperature of the esophagus E measured has reached a predetermined temperature during the surgical ablation of the left atrium. This helps to avoid the possibility of the damage of the esophagus E as described above.

### B. Bending Deformation Operation of Tube Member 21

The catheter 3 according to an example embodiment helps to more reliably prevent the possibility of the damage of the esophagus E upon measuring the internal temperature of the esophagus E as described later in greater detail, by utilizing the bending deformation operation of the region in the vicinity of the tip end of the tube member 21 of the catheter device 2. For example, in a case where the internal temperature of the esophagus E measured has reached a predetermined temperature as described above, the catheter device 2 may be attached to the catheter body 1 described above and the catheter body 1 may be used with the catheter device 2 being integrated therewith to thereby more reliably prevent the possibility of the damage of the esophagus E. In the following, the bending deformation operation of the region in the vicinity of the tip end of the tube member 21 will be described in detail.

First, in a case where an operator performs the rotating operation described above on the handle 22 of the catheter device 2, i.e., the rotary member 221 of the handle 22, as denoted by the broken-line arrow d31 illustrated in FIG. 9A, the drive member 223 may move inside the handle body 220 as denoted by the broken-line arrow d32 illustrated in FIG. 9B in response to the rotating operation. For example, the drive member 223 may move toward the rotary member 221, i.e., toward the base end, in the Z-axis direction inside the handle body 220. Thus, the operating wire 40 may be pulled toward the base end as denoted by the broken-line arrow d4 illustrated in FIG. 9B, allowing the operating wire 40 to be protruded from the opening 210 in the region in the vicinity of the tip end of the tube member 21. It should be noted here that the tip end of the operating wire 40 may be fixed to the tip-end part of the tube member 21 as described above. Accordingly, in a case where the operating wire 40 is pulled toward the base end, the region in the vicinity of the tip end of the tube member 21 may be subjected to the bending deformation in a region around the opening 210 where deformation is easier to occur than any other part of the tube member 21. As a result of the bending deformation, the region in the vicinity of the tip end of the tube member 21 is pressed against a wall surface of the main lumen 61 of the catheter shaft 11 as denoted by a broken-line arrow d5 illustrated in FIG. 7B.

As illustrated in FIG. 7B, for example, the region in the vicinity of the tip end of the tube member 21 is pressed against the wall surface of the main lumen 61 of the catheter shaft 11, causing the region in the vicinity of the tip end of the catheter shaft 11, i.e., the tip-flexible part 11A, to be displaced or subjected to bending displacement as denoted by a broken-line arrow d6 illustrated in FIG. 7B.

As illustrated in FIG. 10B, for example, the displacement of the region in the vicinity of the tip end of the catheter shaft 11 may impart a pressing force, derived from the displacement of the region in the vicinity of the tip end of the catheter shaft 11, against the medial wall of the esophagus E of the patient 9 as denoted by the broken-line arrow d6. The pressing force thus imparted may cause the esophagus E itself of the patient 9 to be displaced as well, as denoted by a broken-line arrow d7. It should be noted that an amount of displacement of the esophagus E itself may be about several centimeters, for example.

### C. Workings and Effects

It is possible for the catheter 3, i.e., the catheter body 1 and the catheter device 2, according to an example embodiment to achieve the following example workings and example effects.

### Bending Deformation Operation of Tube Member 21

In an example embodiment, the bending deformation operation of the region in the vicinity of the tip end of the tube member 21 is carried out by performing the rotating operation on the handle 22 of the catheter device 2 as described above. The tube member 21 having been subjected to the bending deformation displaces the region in the vicinity of the tip end of the catheter shaft 11, causing the esophagus E itself of the patient 9 to be displaced as well, as described above. This configuration helps to allow the esophagus E itself to be distant from a factor that can damage the esophagus E, such as a heating source or a cooling source upon an ablation as described above. For example, it helps to take a measure of shifting a position of the esophagus E to decrease the internal temperature of the esophagus E, in a case where the internal temperature of the esophagus E measured is increased.

Further, in an example embodiment, the catheter device 2 that is a device provided separately from the catheter body 1 is used to displace the region in the vicinity of the tip end of the catheter shaft 11 as described above. This helps to achieve the following example workings in comparison to a comparative example having a configuration, i.e., an integrated configuration, in which operating wires, etc., inserted through a catheter shaft are used to cause a region itself in the vicinity of a tip end of the catheter shaft to be subjected to bending deformation. For example, in an example embodiment, unlike a configuration according to the comparative example described above, no operating wires or the like that serve as a core exist in the catheter shaft 11 in a state in which only the catheter body 1 is first inserted, upon inserting the catheter 3 into the esophagus E through, for example, the nasal cavity N. Accordingly, in an example embodiment, the catheter shaft 11 easily deforms along a shape of, for example, the nasal cavity N as compared with, for example, the comparative example, which helps to reduce a possibility of damaging, for example, the nasal cavity N, including a possibility of bleeding such as nose bleeding. In addition, the tube member 21 of the catheter device 2 is to be inserted through the inside of the catheter shaft 11 even in a case where the tube member 21 is inserted through the main lumen 61 of the catheter shaft 11 after the insertion through, for example, the nasal cavity N as described above, which helps to reduce a possibility of damaging, for example, the nasal cavity N in such a state.

Accordingly, an example embodiment helps to reduce a burden to be imposed on the body of the patient 9 while more reliably preventing a possibility of a damage of the esophagus E, upon measuring the internal temperature of the esophagus E.

In some embodiments, the operating wire 40 inserted through the tube member 21 may be further provided, and the opening 210 having the longitudinal direction in the axial direction, i.e., the Z-axis direction, may be provided in the region in the vicinity of the tip end of the tube member 21. Thus, upon the bending deformation of the region in the vicinity of the tip end of the tube member 21, the region in the vicinity of the tip end of the tube member 21 may be pressed against the wall surface of the main lumen 61 of the catheter shaft 11 with the operating wire 40 being protruded from the opening 210 of the tube member 21 as described above, causing the region in the vicinity of the tip end of the catheter shaft 11 to be displaced. Hence, it helps to achieve, by a simple structure, a mechanism that causes the region in the vicinity of the tip end of the tube member 21 to be subjected to the bending deformation.

In some embodiments, the tube member 21 may be configured by the metal member 70, and at least the base end in the axial direction, i.e., the Z-axis direction, of the metal member 70 may be configured by one or more metal lines that are spirally wound, such as one or more metal coils 71. Thus, at least the base end of the metal member 70 configuring the tube member 21 may be configured by the metal coil 71, which helps to improve a tracking property, or a property that allows for flexible deformation along the shape of the esophagus E, upon the bending deformation of the region in the vicinity of the tip end of the tube member 21. In addition, the tube member 21 becomes easier to deform flexibly, which helps to make it difficult to break upon the bending deformation and helps to improve durability of the tube member 21 as well.

In some embodiments, the metal coil 71 may be provided at the base end of the metal member 70, and the metal coil 71 may not be provided at the tip end of the metal member 70. Thus, the metal coil 71 may be disposed at the base end, which helps improve the tracking property, and the metal coil 71 may not be disposed at the tip end, which, in contrast, helps to decrease the tracking property. Relatively decreasing the tracking property at the tip end helps to effectively displace the esophagus E upon the bending deformation of the region in the vicinity of the tip end of the tube member 21. For example, it helps to prevent the region in the vicinity of the tip end of the catheter shaft 11 from being pushed back by a counteracting force generated upon displacing the esophagus E itself.

In some embodiments, the rigidity k1 of the tube member 21 in the region A1 that is on the tip end side of the region in the vicinity of the tip end of the tube member 21 (i.e., a region near the opening 210), serving as a part to be subjected to the bending deformation, may be greater than the rigidity k2 of the tube member 21 in the region A2 that is on the base end side of the region in the vicinity of the tip end of the tube member 21. Thus, it helps to increase the force of pressing against the wall surface of the main lumen 61 of the catheter shaft 11 upon the bending deformation of the region in the vicinity of the tip end of the tube member 21, which in turn helps to easily displace the region in the vicinity of the tip end of the catheter shaft 11. For example, relatively increasing the rigidity k1 in the vicinity of the region A1 that is on the tip end side of the region in the vicinity of the tip end of the tube member 21, or allowing the region A1 that is on the tip end side of the region in the vicinity of the tip end of the tube member 21 to have a relatively hard structure, helps to increase the force of pressing against the wall surface of the main lumen 61 of the catheter shaft 11. This helps to easily displace the region in the vicinity of the tip end of the catheter shaft 11 and to easily allow the esophagus E to be distant from the factor described above that can damage the esophagus E. Hence, it helps to even more reliably prevent the possibility of the damage of the esophagus E.

### Handles 12 and 22

In an example embodiment, the handle 12 of the catheter body 1 and the handle 22 of the catheter device 2 are configured to be integrated with each other, and configured to be divided into separate members. Thus, it is possible to individually use, depending on a situation, the disposable catheter body 1 and the reusable catheter device 2 as described above, as the catheter 3 as a whole.

Accordingly, an example embodiment helps to reduce a burden to be imposed on the body of the patient 9 while more reliably preventing a possibility of a damage of the esophagus E upon measuring the internal temperature of the esophagus E, and to reduce a cost upon use of the catheter 3.

In some embodiments, the orientation of the deformation upon the bending deformation of the region in the vicinity of the tip end of the tube member 21 may be adjustable, on the basis of the angle of integration upon the integration of the handles 12 and 22 with each other. Thus, it helps to improve a convenience upon the use of the catheter 3.

In some embodiments, the handles 12 and 22 may be configured not to be completely integrated with each other, i.e., may be so disposed as to be separated away from each other by a short distance. Thus, it helps to allow for a fine adjustment of a position of the base end of the operating wire 40 inside the handle 22. Hence, it helps to allow for a fine adjustment of a position of deformation upon the bending deformation of the region in the vicinity of the tip end of the tube member 21 as well, and to improve the convenience upon the use of the catheter 3 accordingly.

In some embodiments, the recesses 122 provided on the handle 12 and the projections 222 provided on the handle 22 may be configured to be fitted with each other. In an example case, in contrast, where the handle 12 has projections and the handle 22 has recesses, the projections can get caught by any outside part upon holding the handle 12 alone, i.e., the handle of the catheter body, for use. In some embodiments, however, the recesses 122 may be provided on the handle 12 and the projections 222 may be provided on the handle 22. Thus, it helps to avoid a possibility that the projections can get caught by any outside part upon using the handle 12 of the catheter body 1 alone. Hence, it helps to improve the convenience upon the use of the catheter 3.

In some embodiments, the connector 121 that allows the electrical leads 50 described above to be coupled to the outside may be provided on a side face of the handle 12 at a position that is off the axial direction, i.e., the Z-axis direction, and the tube member 21 may be configured to be inserted through the main lumen 61 of the catheter shaft 11, with the tube member 21 being inserted linearly through the handle 22.

Thus, the tube member 21 may be inserted through the main lumen 61 of the catheter shaft 11 with the tube member 21 being inserted linearly through the handle 22, which helps to make the force of pressing against the wall surface of the main lumen 61 of the catheter shaft 11 difficult to be attenuated upon the bending deformation of the region in the vicinity of the tip end of the tube member 21. Hence, it helps to easily displace the region in the vicinity of the tip end of the catheter shaft 11 and to easily allow the esophagus E to be distant from the factor described above that can damage the esophagus E. Accordingly, it helps to even more reliably prevent the possibility of the damage of the esophagus E.

In addition, the connector 121 may be provided on the side face of the handle 12 at the position that is off the axial direction, i.e., the Z-axis direction. Thus, it helps to stabilize a position at which the handle 12 is placed upon placement of the handle 12 on a table or the like, for example, and to prevent a rotational movement of the handle 12 around the axial direction, i.e., the Z-axis direction. Hence, it helps to improve the convenience upon the use of the catheter 3.

### 2. Modification Examples

A description is given next of some modification examples 1 to 4 of an example embodiment described above. Note that the like elements are denoted with the same reference numerals, and any redundant description thereof will not be described in detail.

### Modification Examples 1

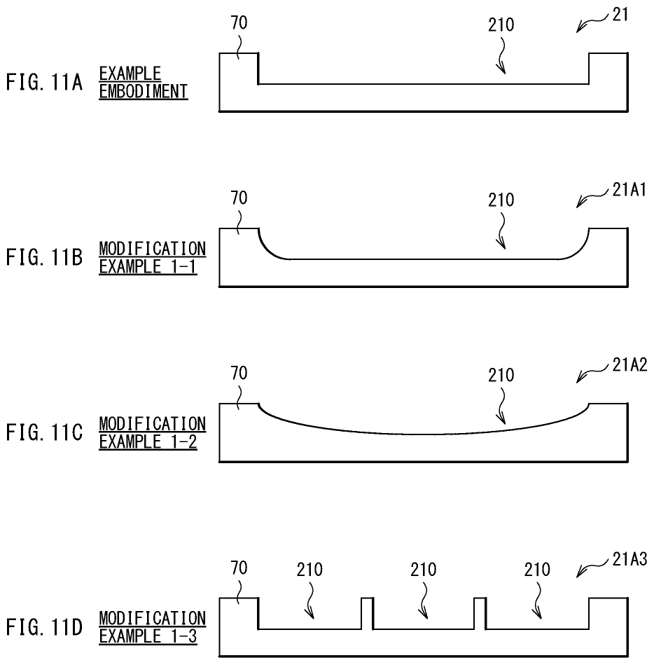

FIGS. 11A to 11D each schematically illustrate an example of a configuration of the opening 210 of a tube member according to any one of an example embodiment or modification examples 1, i.e., modification examples 1-1 to 1-3. FIG. 11A illustrates an example of a configuration of the opening 210 of the tube member 21 according to an example embodiment. FIG. 11B illustrates an example of a configuration of the opening 210 of a tube member 21A1 according to the modification example 1-1. FIG. 11C illustrates an example of a configuration of the opening 210 of a tube member 21A2 according to the modification example 1-2. FIG. 11D illustrates an example of a configuration of the opening 210 of a tube member 21A3 according to the modification example 1-3.

As illustrated in FIG. 11A, the tube member 21 according to an example embodiment may have one opening 210 provided in the region in the vicinity of the tip end of the tube member 21 as described above. The opening 210 may have the longitudinal direction that extends in the axial direction, i.e., the Z-axis direction. The opening 210 may be rectangular in shape, and may have right-angled corners. It should be noted that the right-angled corner does not have to have a complete right angle. In some embodiments, the right-angled corner may be rounded to some extent.

As illustrated in FIG. 11B, the tube member 21A1 according to the modification example 1-1 may have one opening 210 provided in the region in the vicinity of the tip end of the tube member 21A1 as with the tube member 21. The opening 210 may have the longitudinal direction that extends in the axial direction. The opening 210 of the tube member 21A1 may be rectangular in shape, and may have arc-shaped corners.

As illustrated in FIG. 11C, the tube member 21A2 according to the modification example 1-2 may have one opening 210 provided in the region in the vicinity of the tip end of the tube member 21A2. The opening 210 may have the longitudinal direction that extends in the axial direction. The opening 210 of the tube member 21A2 may have curved edges.

As illustrated in FIG. 11D, the tube member 21A3 according to the modification example 1-3 may have the plurality of openings 210 provided in the region in the vicinity of the tip end of the tube member 21A3 and having a similar shape to an example embodiment illustrated in FIG. 11A. The plurality of openings 210 may be provided in the axial direction, i.e., the Z-axis direction. The plurality of openings 210 may be so disposed in the axial direction as to be away from each other.

It is also possible for the modification examples 1, i.e., the modification examples 1-1 to 1-3, having the configurations described above to basically achieve similar effects to an example embodiment by basically similar workings to an example embodiment.

It is also possible for the modification examples 1-1 and 1-2 respectively illustrated in FIGS. 11B and 11C to achieve the following example effects as compared with an example embodiment and the modification example 1-3 respectively illustrated in FIGS. 11A and 11D. For example, the opening 210 according to the modification example 1-1 or 1-2 may have the arc-shaped corners or the curved edges as described above and may thus have a shape that makes it difficult for a stress to be locally concentrated upon the bending deformation of the region in the vicinity of the tip end of the tube member 21A1 or 21A2. Thus, the modification examples 1-1 or 1-2 helps to make it difficult to break even in a case where the tube member 21A1 or 21A2 is repeatedly subjected to the bending deformation. Hence, it helps to improve durability of the tube member 21A1 or 21A2 as compared with an example embodiment or the modification example 1-3.

### Modification Examples 2 and 3

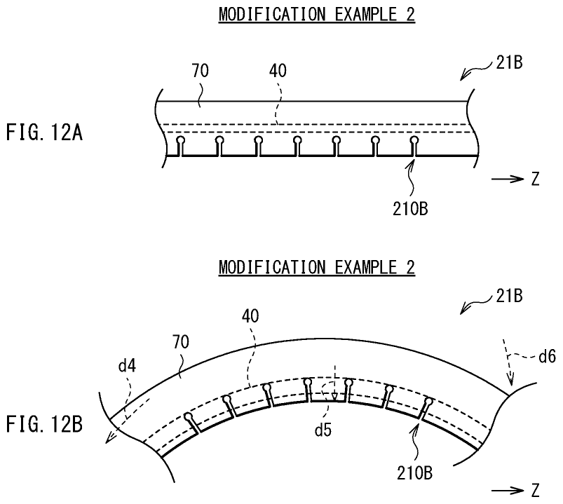

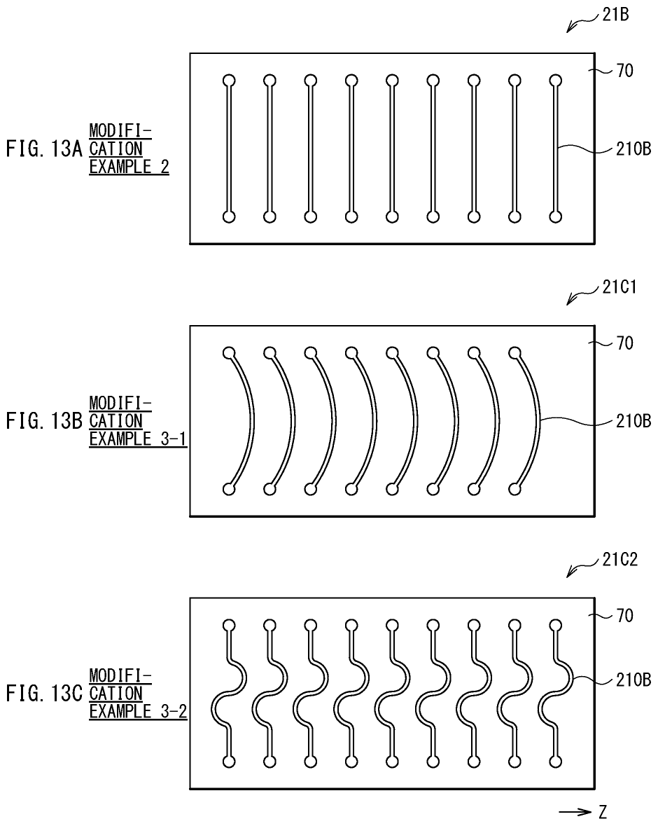

FIGS. 12A and 12B each schematically illustrate an example of a configuration of slits 210B of a tube member 21B according to modification example 2. FIGS. 13A to 13C each schematically illustrate an example of a configuration of the slits 210B according to any one of the modification example 2 or modification examples 3, i.e., the modification examples 3-1 and 3-2, where the slits 210B are deployed on a plane. FIG. 13A schematically illustrates an example of a configuration of the slits 210B of the tube member 21B according to the modification example 2, where the slits 210B are deployed on a plane. FIG. 13B schematically illustrates an example of a configuration of the slits 210B of a tube member 21C1 according to modification example 3-1, where the slits 210B are deployed on a plane. FIG. 13C schematically illustrates an example of a configuration of the slits 210B of a tube member 21C2 according to modification example 3-2, where the slits 210B are deployed on a plane.

As illustrated in FIGS. 12A, 12B, and 13A, the plurality of slits 210B may be provided in the region in the vicinity of the tip end of the tube member 21B according to the modification example 2. The plurality of slits 210B may partially extend in a circumferential direction of the tube member 21B, and may be provided in the axial direction, i.e., the Z-axis direction. As illustrated in FIG. 13A, the plurality of slits 210B in the modification example 2 each may be linear, where the slits 210B are deployed on a plane.

As illustrated in FIG. 13B, the plurality of slits 210B of the tube member 21C1 according to the modification example 3-1 each may be curved, where the slits 210B are deployed on a plane. As illustrated in FIG. 13C, the plurality of slits 210B of the tube member 21C2 according to the modification example 3-2 each may have a hook shape, where the slits 210B are deployed on a plane.

In the modification examples 2 and 3 each having the configuration described above, the region in the vicinity of the tip end of the tube member 21B, 21C1, or 21C2 may be subjected to the bending deformation as follows, as illustrated by way of example in FIG. 12B. For example, the tip end of the operating wire 40 may be fixed to the tip-end part of, for example, the tube member 21B as with an example embodiment. Accordingly, in a case where the operating wire 40 is pulled toward the base end, the region in the vicinity of the tip end of, for example, the tube member 21B may be subjected to the bending deformation in a region around the slits 210B where deformation is easier to occur than any other part of, for example, the tube member 21B. As a result of the bending deformation, the region in the vicinity of the tip end of, for example, the tube member 21B may be pressed against the wall surface of the main lumen 61 of the catheter shaft 11 as denoted by the broken-line arrow d5, as with an example embodiment. Thus, the region in the vicinity of the tip end of the catheter shaft 11 may be displaced as denoted by the broken-line arrow d6, as with an example embodiment. Hence, the modification examples 2 and 3 each help to achieve, by a simple structure, a mechanism that causes the region in the vicinity of the tip end of the tube member 21B, 21C1, or 21C2 to be subjected to the bending deformation.

It is also possible for the modification examples 3-1 and 3-2 respectively illustrated in FIGS. 13B and 13C to achieve the following example effects as compared with the modification example 2 illustrated in FIG. 13A. For example, the slits 210B of the modification example 3-1 or 3-2 having the shape described above helps to suppress a twist of the tube member 21C1 or 21C2 as compared with the modification example 2, even in a case where a force that twists the tube member 21C1 or 21C2 in a circumferential direction of the tube member 21C1 or 21C2 acts on the tube member 21C1 or 21C2.

### Modification Examples 4

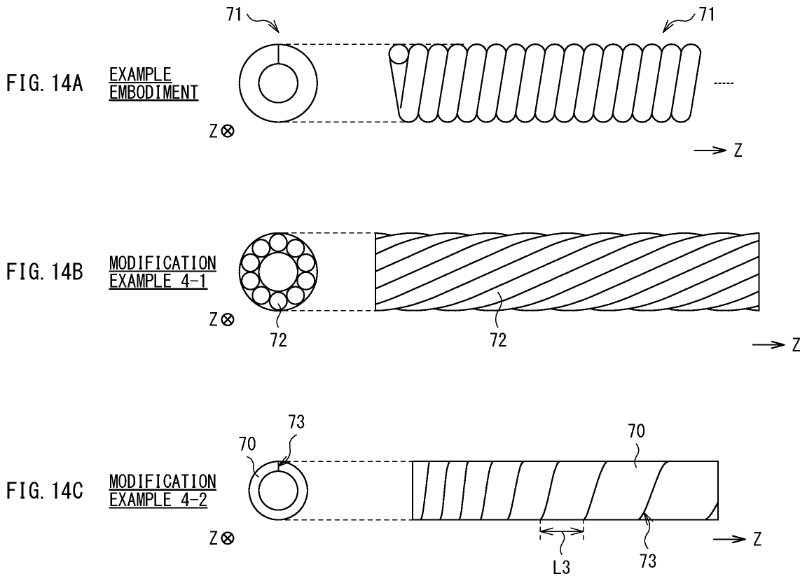

FIGS. 14A to 14C each schematically illustrate an example of a configuration of a metal member 70 of the tube member according any one of an example embodiment or modification examples 4, i.e., modification examples 4-1 and 4-2. FIG. 14A schematically illustrates an example of a configuration of the metal coil 71 described above of the tube member 21 according to an example embodiment. FIG. 14B schematically illustrates an example of a configuration of a metal wire 72 of the tube member according to the modification example 4-1. FIG. 14C schematically illustrates an example of a configuration of a slit 73, etc., of the tube member according to the modification example 4-2.

As illustrated in FIG. 14A, in an example embodiment, a portion in the axial direction, i.e., the Z-axis direction, of the metal member 70 configuring the tube member 21 may have the single metal coil 71 that serves as one or more spirally wound metal lines as described above.

As illustrated in FIG. 14B, in the modification example 4-1, the metal line described above may be a plurality of metal wires 72, or a hollow wire, instead of the single metal coil 71. The plurality of metal wires 72 may be spirally wound.

As illustrated in FIG. 14C, in the modification example 4-2, the metal line described above may have the following example configuration instead of the single metal coil 71. For example, in the modification example 4-2, the metal member 70 may be a single metal line having a spirally formed slit 73 and thus wound spirally. The slit 73 may be formed by laser processing, for example. It should be noted that intervals L3 in the axial direction, i.e., the Z-axis direction, between the slits 73 illustrated in FIG. 14C may have the same value as each other, or may have different values from each other.

It is also possible for the modification examples 4, i.e., the modification examples 4-1 and 4-2, having the configurations described above to basically achieve similar effects to an example embodiment by basically similar workings to an example embodiment.

It is also possible for an example embodiment illustrated in FIG. 14A to achieve the following example effects as compared with the modification examples 4-1 and 4-2 respectively illustrated in FIGS. 14B and 14C. For example, in a case where the operating wire 40 is pulled, a compressing force may be applied to the tube member 21 in the axial direction, i.e., the Z-axis direction, relatively; however, the metal member 70 according to an example embodiment makes it difficult to be deformed in the Z-axis direction upon the application of the compressing force. Hence, an example embodiment helps to more effectively convert a force derived from the pulling of the operating wire 40 into a force that cause the region in the vicinity of the tip end of the tube member 21 to be subjected to the bending deformation as compared with the modification examples 4-1 and 4-2.

### 3. Other Modification Examples

Although the technology has been described with reference to some example embodiments and modification examples, the technology is not limited to such embodiments and modification examples and may be modified in a wide variety of ways.

For example, shapes, locations, characteristics including rigidity characteristics, materials, etc., of the respective members described in the foregoing example embodiments and modification examples are non-limiting, and may respectively be any other shape, location, characteristic, material, etc.

Although the catheter shaft 11 has been described with specific reference to the configuration thereof in the foregoing example embodiments and modification examples, it is not necessary for the catheter shaft 11 to include all of the components. Alternatively, the catheter shaft 11 may be further provided with any other component. For example, factors such as locations, shapes, and the number of electrodes 111 to 115 and the tip 110 of the catheter shaft 11 are not limited to those referred to in the foregoing example embodiments and modification examples. Further, the number of temperature sensors and the number of electrical leads 50 are both not limited to those, i.e., five, described in the foregoing example embodiments and modification examples, and may be adjusted within an example range from one to 20 on an as-needed basis. In some embodiments, the number of temperature sensors and the number of electrical leads 50 both may be two or more, or about four or more. In addition, the foregoing example embodiments and modification examples have been described by referring to an example in which no temperature sensor is electrically coupled to the tip 110; however, this is non-limiting and the temperature sensor may also be electrically coupled to the tip 110 to allow the tip 110 to have a function of measuring the temperature as well. Each of the temperature sensors, including the previously mentioned sensor, is not limited to a configuration described in the foregoing example embodiments and modification examples in which the thermocouple is used, and may utilize other sensors such as a thermistor. The electrodes 111 to 115 and the temperature sensors 51 and 55 do not necessarily have to be electrically coupled.

Although the tube member of the catheter device 2 has been described with specific reference to the configuration thereof in the foregoing example embodiments and modification examples, it is not necessary for the tube member to include all of the components. Alternatively, the tube member may be further provided with any other component. For example, in the foregoing example embodiments and modification examples, the opening or the slits may be provided in the region in the vicinity of the tip end of the tube member, and the operating wire 40 inserted through the tube member may be provided. The technology, however, is not limited thereto. In some embodiments, any other method or configuration may be used to cause the region in the tip end of the tube member to be subjected to the bending deformation. In the foregoing example embodiments and modification examples, the tube member may be configured by the metal member. The technology, however, is not limited thereto. In some embodiments, the tube member may be configured by a non-metal member.

Although the two handles 12 and 22 have been described with specific reference to the configurations thereof in the foregoing example embodiments and modification examples, it is not necessary for each of the handles 12 and 22 to include all of the components. Alternatively, the handles 12 and 22 each may be further provided with any other component. The “deformation operating member” of the handle 22 is not limited to the configuration described in the foregoing example embodiments and modification examples. In some embodiments, any other member other than or in addition to the member described in the foregoing example embodiments and modification examples may be used to configure the “deformation operating member” according to one embodiment of the disclosure.

In the foregoing example embodiments and modification examples, the hollow organ inside the body of the patient may be the esophagus, and the catheter may be used to measure the internal temperature of the esophagus upon performing the surgical ablation of the left atrium on the patient. The technology, however, is not limited thereto. Any embodiment of the technology may be applicable to a catheter to be used for a measurement of an internal temperature of any hollow organ inside the body other than the esophagus.

Furthermore, the technology encompasses any possible combination of some or all of the various example embodiments and the modification examples described herein and incorporated herein.

It is possible to achieve at least the following configurations from the above-described example embodiments of the disclosure.

(1)

A catheter device to be applied to a catheter, the catheter including a catheter shaft, and a plurality of temperature sensors provided in a region in the vicinity of a tip end of the catheter shaft and configured to measure an internal temperature of a hollow organ inside the body, the catheter device including:
a tube member extending in an axial direction, and configured to be inserted through a lumen provided in the catheter shaft; and
a second handle provided on a base end of the tube member, and including a deformation operating member configured to receive an operation that causes a region in the vicinity of a tip end of the tube member to be subjected to bending deformation, the second handle being provided separately from a first handle provided on a base end of the catheter shaft, in which, upon the bending deformation of the region in the vicinity of the tip end of the tube member in response to the operation of the deformation operating member, the tube member to be subjected to the bending deformation is configured to be pressed against a wall surface of the lumen of the catheter shaft to displace the region in the vicinity of the tip end of the catheter shaft, and
the second handle is configured to be integrated with the first handle.

In the catheter device according to one embodiment of the technology, in a case where the operation that causes the region in the vicinity of the tip end of the tube member, configured to be inserted through the lumen of the catheter shaft, of the catheter device to be subjected to the bending deformation is performed on the deformation operating member of the second handle of the catheter device, the tube member to be subjected to the bending deformation is configured to be pressed against the wall surface of the lumen of the catheter shaft to displace the region in the vicinity of the tip end of the catheter shaft, upon the bending deformation of the region in the vicinity of the tip end of the tube member. Thus, a pressing force derived from the displacement of the region in the vicinity of the tip end of the catheter shaft is imparted against the medial wall of the hollow organ inside the body, causing the hollow organ itself to be displaced as well. Hence, it helps to allow the hollow organ itself to be distant from a factor that can damage the hollow organ, such as a heating source or a cooling source upon an ablation. Further, the catheter device that is a device provided separately from the catheter body is used to displace the region in the vicinity of the tip end of the catheter shaft. This helps to achieve the following example workings in comparison to, for example, a case having a configuration, i.e., an integrated configuration, in which operating wires, etc., inserted through a catheter shaft are used to cause a region itself in the vicinity of a tip end of the catheter shaft to be subjected to bending deformation. For example, unlike the integrated configuration described above, no operating wires or the like that serve as a core exist in the catheter shaft in a state in which only the catheter body is first inserted, upon inserting the catheter into the hollow organ inside the body through, for example, the nasal cavity. Accordingly, the catheter shaft easily deforms along a shape of, for example, the nasal cavity as compared with, for example, the integrated configuration, which helps to reduce a possibility of damaging, for example, the nasal cavity, including a possibility of bleeding such as nose bleeding. In addition, the tube member of the catheter device is to be inserted through the inside of the catheter shaft even in a case where the tube member is inserted through the lumen of the catheter shaft after the insertion through, for example, the nasal cavity as described above, which helps to reduce a possibility of damaging, for example, the nasal cavity in such a state. Further, the first handle of the catheter body and the second handle of the catheter device are configured to be integrated with each other, and configured to be divided into separate members. Thus, it is possible to individually use the disposable catheter body and the reusable catheter device depending on a situation.

(2)

The catheter device according to (1), in which
the second handle has a projection, and
the projection of the second handle is configured to be fitted with a recess provided on the first handle.

In an example case, in contrast, where the first handle has the projection and the second handle has the recess, the projection can get caught by any outside part upon holding the first handle alone, i.e., the handle of the catheter body, for use. In some embodiments, however, the recess may be provided on the first handle and the projection may be provided on the second handle. Thus, it helps to avoid a possibility that the projection can get caught by any outside part upon using the handle of the catheter body alone. Hence, it helps to improve the convenience upon the use of the catheter.

(3)

The catheter device according to (1) or (2), in which an orientation of deformation upon the bending deformation of the region in the vicinity of the tip end of the tube member is adjustable, on the basis of an angle of integration, within a plane orthogonal to the axial direction, upon the integration of the first handle and the second handle with each other.

With this configuration, the orientation of the bending deformation of the tube member may be adjustable on the basis of the angle of integration. Thus, it helps to improve a convenience upon the use of the catheter.

(4)

A catheter body of a catheter, the catheter being configured to measure an internal temperature of a hollow organ inside the body, the catheter body including:
a catheter shaft having a lumen;
a plurality of temperature sensors provided in a region in the vicinity of a tip end of the catheter shaft, and configured to measure the internal temperature of the hollow organ inside the body; and
a first handle provided on a base end of the catheter shaft,
in which, upon bending deformation of a region in the vicinity of a tip end of a tube member of a catheter device in response to an operation of a deformation operating member of the catheter device, the tube member to be subjected to the bending deformation is configured to be pressed against a wall surface of the lumen of the catheter shaft to displace the region in the vicinity of the tip end of the catheter shaft, the catheter device including
   the tube member extending in an axial direction, and configured to be inserted through the lumen of the catheter shaft, and
   a second handle provided on a base end of the tube member, and including the deformation operating member configured to receive the operation that causes the region in the vicinity of the tip end of the tube member to be subjected to the bending deformation, the second handle being provided separately from the first handle, and
the first handle is configured to be integrated with the second handle.

In the catheter body according to one embodiment of the technology, in a case where the operation that causes the region in the vicinity of the tip end of the tube member, configured to be inserted through the lumen of the catheter shaft, of the catheter device to be subjected to the bending deformation is performed on the deformation operating member of the second handle of the catheter device, the tube member to be subjected to the bending deformation is configured to be pressed against the wall surface of the lumen of the catheter shaft to displace the region in the vicinity of the tip end of the catheter shaft, upon the bending deformation of the region in the vicinity of the tip end of the tube member. Thus, a pressing force derived from the displacement of the region in the vicinity of the tip end of the catheter shaft is imparted against the medial wall of the hollow organ inside the body, causing the hollow organ itself to be displaced as well. Hence, it helps to allow the hollow organ itself to be distant from a factor that can damage the hollow organ, such as a heating source or a cooling source upon an ablation. Further, the catheter device that is a device provided separately from the catheter body is used to displace the region in the vicinity of the tip end of the catheter shaft. This helps to achieve the following example workings in comparison to, for example, a case having a configuration, i.e., an integrated configuration, in which operating wires, etc., inserted through a catheter shaft are used to cause a region itself in the vicinity of a tip end of the catheter shaft to be subjected to bending deformation. For example, unlike the integrated configuration described above, no operating wires or the like that serve as a core exist in the catheter shaft in a state in which only the catheter body is first inserted, upon inserting the catheter into the hollow organ inside the body through, for example, the nasal cavity. Accordingly, the catheter shaft easily deforms along a shape of, for example, the nasal cavity as compared with, for example, the integrated configuration, which helps to reduce a possibility of damaging, for example, the nasal cavity, including a possibility of bleeding such as nose bleeding. In addition, the tube member of the catheter device is to be inserted through the inside of the catheter shaft even in a case where the tube member is inserted through the lumen of the catheter shaft after the insertion through, for example, the nasal cavity as described above, which helps to reduce a possibility of damaging, for example, the nasal cavity in such a state. Further, the first handle of the catheter body and the second handle of the catheter device are configured to be integrated with each other, and configured to be divided into separate members. Thus, it is possible to individually use the disposable catheter body and the reusable catheter device depending on a situation.

(5)

The catheter body according to (4), in which
the first handle has a recess, and
the recess of the first handle is configured to be fitted with a projection provided on the second handle.

With this configuration, the recess may be provided on the first handle and the projection may be provided on the second handle. Thus, it helps to avoid a possibility that the projection can get caught by any outside part upon using the first handle of the catheter body alone. Hence, it helps to improve the convenience upon the use of the catheter.

(6)

The catheter body according to (4) or (5), further including a connector provided on a side face of the first handle at a position that is off the axial direction, and configured to allow electrical leads electrically coupled to the temperature sensors to be coupled to outside, in which
the tube member is configured to be inserted through the lumen of the catheter shaft, with the tube member being inserted linearly through the first handle.

With this configuration, the tube member may be inserted through the lumen of the catheter shaft with the tube member being inserted linearly through the first handle, which helps to make the force of pressing against the wall surface of the lumen of the catheter shaft difficult to be attenuated upon the bending deformation of the region in the vicinity of the tip end of the tube member. Hence, it helps to easily displace the region in the vicinity of the tip end of the catheter shaft and to easily allow the hollow organ inside the body to be distant from the factor described above that can damage the hollow organ. Accordingly, it helps to even more reliably prevent the possibility of the damage of the hollow organ. In addition, the connector may be provided on the side face of the first handle at the position that is off the axial direction. Thus, it helps to stabilize a position at which the first handle is placed upon placement of the first handle on a table or the like, for example, and to prevent a rotational movement of the first handle around the axial direction. Hence, it helps to improve the convenience upon the use of the catheter.

(7)

A catheter configured to measure an internal temperature of a hollow organ inside the body, the catheter including:
a catheter body; and
a catheter device,
the catheter body including
   a catheter shaft having a lumen,
   a plurality of temperature sensors provided in a region in the vicinity of a tip end of the catheter shaft, and configured to measure the internal temperature of the hollow organ inside the body, and
   a first handle provided on a base end of the catheter shaft, the catheter device including
   a tube member extending in an axial direction, and inserted through the lumen of the catheter shaft, and
   a second handle provided on a base end of the tube member, and including a deformation operating member configured to receive an operation that causes a region in the vicinity of a tip end of the tube member to be subjected to bending deformation,
in which, upon the bending deformation of the region in the vicinity of the tip end of the tube member in response to the operation of the deformation operating member, the tube member to be subjected to the bending deformation is configured to be pressed against a wall surface of the lumen of the catheter shaft to displace the region in the vicinity of the tip end of the catheter shaft, and
the first handle and the second handle are configured to be integrated with each other.

In the catheter according to one embodiment of the technology, in a case where the operation that causes the region in the vicinity of the tip end of the tube member, inserted through the lumen of the catheter shaft, of the catheter device to be subjected to the bending deformation is performed on the deformation operating member of the second handle of the catheter device, the tube member to be subjected to the bending deformation is configured to be pressed against the wall surface of the lumen of the catheter shaft to displace the region in the vicinity of the tip end of the catheter shaft, upon the bending deformation of the region in the vicinity of the tip end of the tube member. Thus, a pressing force derived from the displacement of the region in the vicinity of the tip end of the catheter shaft is imparted against the medial wall of the hollow organ inside the body, causing the hollow organ itself to be displaced as well. Hence, it helps to allow the hollow organ itself to be distant from a factor that can damage the hollow organ, such as a heating source or a cooling source upon an ablation. Further, the catheter device that is a device provided separately from the catheter body is used to displace the region in the vicinity of the tip end of the catheter shaft. This helps to achieve the following example workings in comparison to, for example, a case having a configuration, i.e., an integrated configuration, in which operating wires, etc., inserted through a catheter shaft are used to cause a region itself in the vicinity of a tip end of the catheter shaft to be subjected to bending deformation. For example, unlike the integrated configuration described above, no operating wires or the like that serve as a core exist in the catheter shaft in a state in which only the catheter body is first inserted, upon inserting the catheter into the hollow organ inside the body through, for example, the nasal cavity. Accordingly, the catheter shaft easily deforms along a shape of, for example, the nasal cavity as compared with, for example, the integrated configuration, which helps to reduce a possibility of damaging, for example, the nasal cavity, including a possibility of bleeding such as nose bleeding. In addition, the tube member of the catheter device is to be inserted through the inside of the catheter shaft even in a case where the tube member is inserted through the lumen of the catheter shaft after the insertion through, for example, the nasal cavity as described above, which helps to reduce a possibility of damaging, for example, the nasal cavity in such a state. Further, the first handle of the catheter body and the second handle of the catheter device are configured to be integrated with each other, and configured to be divided into separate members. Thus, it is possible to individually use the disposable catheter body and the reusable catheter device depending on a situation.

(8)

The catheter according to (7), in which
the hollow organ inside the body includes esophagus, and
the catheter is configured to measure an internal temperature of the esophagus upon surgical ablation of left atrium of a patient.

In the catheter device, the catheter body, or the catheter according to at least one embodiment of the technology, upon the bending deformation of the region in the vicinity of the tip end of the tube member, the tube member to be subjected to the bending deformation is configured to be pressed against the wall surface of the lumen of the catheter shaft to displace the region in the vicinity of the tip end of the catheter shaft. Thus, it helps to cause the hollow organ itself inside the body to be displaced as well. Hence, it helps to allow the hollow organ itself to be distant from a factor that can damage the hollow organ. In addition, the catheter device that is a device provided separately from the catheter body is used to displace the region in the vicinity of the tip end of the catheter shaft. This helps to reduce a possibility of damaging, for example, the nasal cavity of a patient as compared with, for example, the integrated configuration described above. Further, the first handle and the second handle are configured to be integrated with each other. Thus, it is possible to individually use the disposable catheter body and the reusable catheter device depending on a situation. Accordingly, at least one embodiment of the technology helps to reduce a burden to be imposed on the body of a patient while more reliably preventing a possibility of a damage of a hollow organ inside the body upon measuring an internal temperature of the hollow organ, and to reduce a cost upon use.

Although the technology has been described in terms of exemplary embodiments, it is not limited thereto. It should be appreciated that variations may be made in the described embodiments by persons skilled in the art without departing from the scope of the technology as defined by the following claims. The limitations in the claims are to be interpreted broadly based on the language employed in the claims and not limited to examples described in this specification or during the prosecution of the application, and the examples are to be construed as non-exclusive. For example, in this disclosure, the use of the terms first, second, etc., do not denote any order or importance, but rather the terms first, second, etc., are used to distinguish one element from another. The term “disposed on/provided on/formed on” and its variants as used herein refer to elements disposed directly in contact with each other or indirectly by having intervening structures therebetween. Moreover, no element or component in this disclosure is intended to be dedicated to the public regardless of whether the element or component is explicitly recited in the following claims.

What is claimed is:

1. A catheter device to be applied to a catheter, the catheter including a catheter shaft, and a plurality of temperature sensors provided in a region in vicinity of a tip end of the catheter shaft and configured to measure an internal temperature of a hollow organ inside body, the catheter device comprising:
a tube member extending in an axial direction, and configured to be inserted through a lumen provided in the catheter shaft; and
a second handle provided on a base end of the tube member, and including a deformation operating member configured to receive an operation that causes a region in vicinity of a tip end of the tube member to be subjected to bending deformation, the second handle being provided separately from a first handle provided on a base end of the catheter shaft,
wherein, upon the bending deformation of the region in the vicinity of the tip end of the tube member in response to the operation of the deformation operating member, the tube member to be subjected to the bending deformation is configured to be pressed against a wall surface of the lumen of the catheter shaft to displace the region in the vicinity of the tip end of the catheter shaft, and
the second handle is configured to be integrated with the first handle.

2. The catheter device according to claim 1, wherein
the second handle has a projection, and
the projection of the second handle is configured to be fitted with a recess provided on the first handle.

3. The catheter device according to claim 1, wherein an orientation of deformation upon the bending deformation of the region in the vicinity of the tip end of the tube member is adjustable, on a basis of an angle of integration, within a plane orthogonal to the axial direction, upon the integration of the first handle and the second handle with each other.

4. A catheter body of a catheter, the catheter being configured to measure an internal temperature of a hollow organ inside body, the catheter body comprising:
a catheter shaft having a lumen;
a plurality of temperature sensors provided in a region in vicinity of a tip end of the catheter shaft, and configured to measure the internal temperature of the hollow organ inside the body; and
a first handle provided on a base end of the catheter shaft,
wherein, upon bending deformation of a region in vicinity of a tip end of a tube member of a catheter device in response to an operation of a deformation operating member of the catheter device, the tube member to be subjected to the bending deformation is configured to be pressed against a wall surface of the lumen of the catheter shaft to displace the region in the vicinity of the tip end of the catheter shaft, the catheter device including
the tube member extending in an axial direction, and configured to be inserted through the lumen of the catheter shaft, and
a second handle provided on a base end of the tube member, and including the deformation operating member configured to receive the operation that causes the region in the vicinity of the tip end of the tube member to be subjected to the bending deformation, the second handle being provided separately from the first handle, and
the first handle is configured to be integrated with the second handle.

5. The catheter body according to claim 4, wherein
the first handle has a recess, and
the recess of the first handle is configured to be fitted with a projection provided on the second handle.

6. The catheter body according to claim 4, further comprising a connector provided on a side face of the first handle at a position that is off the axial direction, and configured to allow electrical leads electrically coupled to the temperature sensors to be coupled to outside, wherein
the tube member is configured to be inserted through the lumen of the catheter shaft, with the tube member being inserted linearly through the first handle.

7. A catheter configured to measure an internal temperature of a hollow organ inside body, the catheter comprising:
a catheter body; and
a catheter device,
the catheter body including
a catheter shaft having a lumen,
a plurality of temperature sensors provided in a region in vicinity of a tip end of the catheter shaft, and configured to measure the internal temperature of the hollow organ inside the body, and
a first handle provided on a base end of the catheter shaft,
the catheter device including
a tube member extending in an axial direction, and inserted through the lumen of the catheter shaft, and
a second handle provided on a base end of the tube member, and including a deformation operating member configured to receive an operation that causes a region in vicinity of a tip end of the tube member to be subjected to bending deformation,
wherein, upon the bending deformation of the region in the vicinity of the tip end of the tube member in response to the operation of the deformation operating member, the tube member to be subjected to the bending deformation is configured to be pressed against a wall surface of the lumen of the catheter shaft to displace the region in the vicinity of the tip end of the catheter shaft, and
the first handle and the second handle are configured to be integrated with each other.

8. The catheter according to claim 7, wherein
the hollow organ inside the body comprises esophagus, and
the catheter is configured to measure an internal temperature of the esophagus upon surgical ablation of left atrium of a patient.
